(19) United States
(12) Patent Application Publication
(10) Pub. No.: US 2025/0228830 A1
(43) Pub. Date: Jul. 17, 2025

(54) 4-AMINOPYRIDINE (4-AP) AND BONE MORPHOGENETIC PROTEIN 2 (BMP-2)

(71) Applicant: The Penn State Research Foundation, University Park, PA (US)

(72) Inventors: Sandeep Moothedath Subrahmanian, University Park, PA (US); Prem Kumar Govindappa, University Park, PA (US); John Elfar, Hummelstown, PA (US)

(21) Appl. No.: 19/068,442

(22) Filed: Mar. 3, 2025

Related U.S. Application Data
(63) Continuation of application No. 18/140,258, filed on Apr. 27, 2023, now Pat. No. 12,268,677.
(60) Provisional application No. 63/335,349, filed on Apr. 27, 2022.

(51) Int. Cl. A61K 31/4409; A61P 19/08

(52) U.S. Cl. CPC A61K 31/4409; A61P 19/08

(57) ABSTRACT

This document relates to methods and materials for using 4-aminopyridine (4-AP) and/or one or more derivatives of 4-AP. For example, a composition containing 4-AP and/or one or more derivatives of 4-AP can be administered (e.g., orally administered) to a mammal to increase a level of bone morphogenetic protein 2 (BMP-2) polypeptides within the mammal. For example, a composition containing 4-AP and/or one or more derivatives of 4-AP can be administered (e.g., orally administered) to a mammal having bone loss to increase the level of BMP-2 within the mammal, thereby treating the mammal's bone loss.

### CROSS-REFERENCE TO RELATED APPLICATIONS

This application is a continuation of U.S. application Ser. No. 18/140,258, filed Apr. 27, 2023, which claims the benefit of U.S. Patent Application Ser. No. 63/335,349, filed on Apr. 27, 2022. The disclosure of the prior applications are considered part of (and are incorporated by reference in) the disclosure of this application.

### TECHNICAL FIELD

This document relates to methods and materials for using 4-aminopyridine (4-AP) and/or one or more derivatives of 4-AP. For example, a composition containing 4-AP and/or one or more derivatives of 4-AP can be administered (e.g., orally administered) to a mammal to increase a level of bone morphogenetic protein 2 (BMP-2) polypeptides within the mammal. For example, a composition containing 4-AP and/or one or more derivatives of 4-AP can be administered (e.g., orally administered) to a mammal having bone loss to treat the mammal.

### BACKGROUND INFORMATION

BMP-2 polypeptides can stimulate the production of bone, and recombinant human protein (rhBMP-2) polypeptides are currently available for orthopedic and dental uses in the United States (Khan et al., Expert Opin. Biol. Ther., 4(5):741-8 (2004); and Schliephake et al., Clin. Oral Implants Res., 16(5):563-9 (2005)). However, current BMP-2 polypeptide therapies require invasive implantation of carriers and/or delivery systems that provide BMP-2 polypeptides (Agrawal et al., J. Biomed. Mat. Res. Part B: Appl. Biomater., Early View (4):904-925 (2016)).

### SUMMARY

This document provides methods and materials for using 4-AP and/or one or more derivatives of 4-AP. For example, a composition containing 4-AP and/or one or more derivatives of 4-AP can be administered (e.g., orally administered) to a mammal to increase a level of BMP-2 polypeptides within the mammal. For example, a composition containing 4-AP and/or one or more derivatives of 4-AP can be administered (e.g., orally administered) to a mammal having bone loss to increase the level of BMP-2 within the mammal, thereby treating the mammal's bone loss.

As demonstrated herein, administering a composition containing 4-AP and/or one or more derivatives of 4-AP to a mammal (e.g., a human) can increase BMP-2 expression within the mammal. For example, a composition containing 4-AP and/or one or more derivatives of 4-AP can be administered to a mammal (e.g., a human) having a disease, disorder, or condition characterized by a reduced level of one or more BMP-2 polypeptides (e.g., a disease, disorder, or condition associated with bone loss) to treat the mammal.

Having the ability to increase BMP-2 expression as described herein (e.g., by orally administering a composition containing 4-AP and/or one or more derivatives of 4-AP) provides a unique and unrealized opportunity to non-invasively increase a level of BMP-2 polypeptides (e.g., to treat a disease, disorder, or condition associated with bone loss) in a mammal.

In general, one aspect of this document features methods for increasing a level of a BMP-2 polypeptide in a mammal. The methods can include, or consist essentially of, administering a composition comprising 4-AP or one or more derivatives of 4-AP to a mammal. The mammal can be a human. The human can have been identified as being in need of increased BMP-2 polypeptide expression. The administering can include a systemic administration. The systemic administration can be an oral administration. The mammal can have a disease, disorder, or condition associated with reduced BMP-2 polypeptide expression. The disease, disorder, or condition associated with reduced BMP-2 polypeptide expression can include bone loss. The disease, disorder, or condition can be osteoporosis, a bone cancer, a bone fracture, a congenital bone disease, a degenerative bone disease, a dietary bone insufficiency, or an environmental exposure bone insufficiency.

In another aspect, this document features methods for treating a mammal having a disease, disorder, or condition associated with reduced BMP-2 polypeptide expression. The methods can include, or consist essentially of, administering a composition comprising 4-AP or one or more derivatives of 4-AP to mammal having a disease, disorder, or condition associated with reduced BMP-2 polypeptide expression; and detecting an increased level of a BMP-2 polypeptide in a sample obtained from the mammal. The increased level of the BMP-2 polypeptide can be a serum level that is greater than 10 μg/mL. The sample can be a urine, whole blood, serum, plasma, bone tissue, bone marrow, lymph, saliva, or cerebrospinal fluid (CSF) sample. The sample can be obtained at least 0.5 hours after the administering of the composition comprising 4-AP or one or more derivatives of 4-AP to the mammal. The mammal can be a human. The administering can include a systemic administration. The systemic administration can be oral administration. The human can have been identified as having the disease, disorder, or condition associated with reduced BMP-2 polypeptide expression. The disease, disorder, or condition associated with reduced BMP-2 polypeptide expression can include bone loss. The disease, disorder, or condition can be osteoporosis, a bone cancer, a bone fracture, a congenital bone disease, a degenerative bone disease, a dietary bone insufficiency, or an environmental exposure bone insufficiency.

In another aspect, this document features uses of a composition comprising 4-AP or one or more derivatives of 4-AP to increase BMP-2 polypeptide expression in a mammal. The mammal can be a human. The human can have been identified as being in need of increased BMP-2 polypeptide expression.

In another aspect, this document features compositions comprising 4-AP or one or more derivatives of 4-AP for use in the preparation of a medicament to increase BMP-2 polypeptide expression in a mammal. The mammal can be a human. The human can have been identified as being in need of increased BMP-2 polypeptide expression.

In another aspect, this document features compositions comprising 4-AP or one or more derivatives of 4-AP for use in increasing BMP-2 polypeptide expression in a mammal. The mammal can be a human. The human can have been identified as being in need of increased BMP-2 polypeptide expression.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. 4-AP upregulates BMP-2 expression in human bone marrow mesenchymal stem cells (hBMSCs). hBMSCs were incubated in Dulbecco's modified Eagle's medium supplemented with 10% FBS and penicillin/streptomycin (100 U/mL penicillin and 100 mg/mL streptomycin) in the presence of 1000 μM 4-AP induced expression of BMP-2 gene. BMP-2 gene expression was increased at day 3 and remained high at day 7 and day 14. The gene expression levels of BMP-2 were normalized with β-actin and were then used to calculate fold change in 4-AP treated compared to control. Data are presented as mean±SEM (n=3). Values are significant at *p<0.05, **p<0.01. Abbreviations used; BMP-2-bone morphogenetic protein-2.

FIGS. 2A-2B. Western blot analysis of BMP-2 protein. Incubation of hBMSCs in Dulbecco's modified Eagle's medium supplemented with 10% FBS and penicillin/streptomycin (100 U/mL penicillin and 100 mg/mL streptomycin) in the presence of 1000 μM 4-AP increased BMP-2 protein at day 3, 7, and 14. The blots were stripped and reprobed with anti-GAPDH antibody. FIG. 2A: Representative image of a western blot of BMP-2 at day 3, 7, and 14. FIG. 2B: Densitometric analysis was carried out and the BMP-2 was normalized with GAPDH to calculate fold change. Data are presented as mean±SEM (n=3). Values are significant at *p<0.05, Abbreviations used; 4-AP-4-aminopyridine, BMP-2-bone morphogenetic protein-2, GAPDH-glyceraldehyde-3-phosphate dehydrogenase.

FIG. 3. 4-AP upregulates BMP-2 gene expression in human fetal osteoblasts (hFOB). PCR analysis revealed that hFOB treated with 4-AP significantly increased the expression of BMP-2 compared with the control group. hFOB were incubated in Dulbecco's modified Eagle's medium supplemented with 10% FBS and penicillin/streptomycin (100 U/ml penicillin and 100 mg/ml streptomycin) in presence of 1000 μM 4-AP for 3 days induced expression of genes. The gene expression levels were normalized with β-actin and was then used to calculate fold change in 4-AP treated compared to control. Data are presented as mean±SEM (n=3). Values are significant at *p<0.05. Abbreviations used; 4-AP-4-aminopyridine.

FIGS. 4A-4D. 4-AP enhanced bone fracture healing mice. 8 week old C57Bl/6J mice were subjected to mid-diaphysis tibial fracture, treated with 4-AP for 21 days, and harvested. FIG. 4A: Representative X-ray images at the end of 21 days of 4-AP treatment. FIG. 4B: Representative micro-CT images of tibia fracture on day 21. FIG. 4C: Representative cross-sectional micro-CT images of the fracture show less mineralization in untreated. FIG. 4D: Graphical presentation of Micro CT analysis of bone mineral density (BMD), and bone volume density (BV/TV). Data are presented as mean±SEM (n=6). Values are significant at *p<0.05. Abbreviations used; 4-AP-4-aminopyridine.

### DETAILED DESCRIPTION

This document provides methods and materials for using 4-AP and/or one or more derivatives of 4-AP. In some cases, this document provides methods and materials for increasing level of one or more BMP-2 polypeptides within a mammal (e.g., a human). For example, a composition containing 4-AP and/or one or more derivatives of 4-AP can be administered (e.g., orally administered) to a mammal to increase a level of BMP-2 polypeptides within the mammal. In some cases, this document provides methods and materials for treating a mammal (e.g., a human) identified as being in need of increased BMP-2 polypeptide expression (e.g., having a disease, disorder, or condition characterized by a reduced level of one or more BMP-2 polypeptides). For example, a composition containing 4-AP and/or one or more derivatives of 4-AP can be administered (e.g., orally administered) to a mammal having bone loss to increase the level of BMP-2 within the mammal, thereby treating the mammal's bone loss.

Any appropriate mammal can be treated as described herein. Examples of mammals that can be treated as described herein include, without limitation, humans, non-human primates such as monkeys, horses, bovine species, porcine species, dogs, cats, mice, rats, rabbits, and goats. In some cases, a human in need of increased BMP-2 polypeptide expression can be treated as described herein. In some cases, a human having bone loss (or identified as being likely to experience bone loss) can be treated as described herein. In some cases, the methods provided herein can be used to treat an animal that is not a mammal. For example, the methods provided herein can be used to increase a level of one or more BMP-2 polypeptides in a fowl (e.g., a chicken).

In some cases, a mammal (e.g., a human) to be treated as described herein (e.g., by administering a composition containing 4-AP and/or one or more derivatives of 4-AP) can have bone loss in one or more injured bones (e.g., can have injury-induced bone loss). For example, a mammal having nerve injury-induced bone loss can be treated by administering a composition containing 4-AP and/or one or more derivatives of 4-AP. For example, a mammal having muscle injury-induced bone loss can be treated by administering a composition containing 4-AP and/or one or more derivatives of 4-AP.

In some cases, a mammal (e.g., a human) to be treated as described herein (e.g., by administering a composition containing 4-AP and/or one or more derivatives of 4-AP) can have one or more diseases, disorders, or conditions associated with bone loss. Examples of diseases, disorders, and conditions associated with bone loss include, without limitation, osteoporosis, bone cancers, bone fractures (e.g., insufficiency fractures), congenital bone diseases, degenerative bone diseases, dietary bone insufficiencies, and environmental exposure bone insufficiencies (e.g., bone insufficiencies caused by toxic exposure such as lead poisoning).

In some cases, a mammal (e.g., a human) to be treated as described herein (e.g., by administering a composition containing 4-AP and/or one or more derivatives of 4-AP) can be in need of bone fusion. For example, a human who has been identified as being in need of bone fusion (e.g., a mammal having one or more bone fractures) can be treated as described herein (e.g., by administering a composition containing 4-AP and/or one or more derivatives of 4-AP).

In some cases, a mammal (e.g., a human) having bone loss (or identified as being likely to experience bone loss) that lacks bone fractures can be treated as described herein (e.g., by administering a composition containing 4-AP and/or one or more derivatives of 4-AP). For example, a human with no current bone fractures who has been identified as having bone loss can be treated as described herein (e.g., by administering a composition containing 4-AP and/or one or more derivatives of 4-AP).

In some cases, a composition containing 4-AP and/or one or more derivatives of 4-AP can be administered (e.g., orally administered) to a mammal (e.g., a human such as a human identified as being in need of increased BMP-2 polypeptide expression) to increase a level of one or more BMP-2 polypeptides within the mammal. For example, a composition containing 4-AP and/or one or more derivatives of 4-AP can be administered (e.g., orally administered) to a mammal (e.g., a human) in need thereof (e.g., a mammal identified as being in need of increased BMP-2 polypeptide expression) to increase expression of one or more BMP-2 polypeptides in cells within the mammal. The term “increased level” as used herein with respect to a level of a BMP-2 polypeptide in a mammal refers to any level that is greater than the level of that BMP-2 polypeptide observed in that mammal prior to being treated as described herein (e.g., prior to being administered a composition containing 4-AP and/or one or more derivatives of 4-AP). In some cases, an increased level of a BMP-2 polypeptide can be a level that is at least 5 percent (e.g., at least 10, at least 15, at least 20, at least 25, at least 35, at least 50, at least 75, at least 100, or at least 150 percent) greater than the level of that BMP-2 polypeptide prior to being treated as described herein. In some cases, an increased level of a BMP-2 polypeptide can be a serum level that is greater than 10 μg/mL in a human. In some cases, when samples have an undetectable level of a BMP-2 polypeptide prior to treatment as described herein, an increased level can be any detectable level of a BMP-2 polypeptide. It will be appreciated that levels from comparable samples are used when determining whether or not a particular level is an increased level.

When the methods and materials described herein are used to increase a level of one or more BMP-2 polypeptides within a mammal (e.g., a human such as a human identified as being in need of increased BMP-2 polypeptide expression), a level of any appropriate BMP-2 polypeptide can be increased. Examples of BMP-2 polypeptides that can be increased as described herein include, without limitation, BMP-2 polypeptides having an amino acid sequence set forth in any one of National Center for Biotechnology Information (NCBI) GenBank® or GenPept® Accession No. P12643, a fragment of this amino acid sequence (e.g., as in an amino acid sequence including amino acids 283-396 of the sequence set forth as Accession No. P12643), or a homodimer of the fragment (e.g., a disulfide-linked homodimer in which each monomer of the homodimer includes an amino acid sequence having amino acids 283-396 of the sequence set forth as Accession No. P12643). BMP-2 polypeptides can be used in any form for the methods herein, such as pro-peptide forms, cleaved or mature forms, or dimerized forms, as may be present in a sample obtained from a mammal.

When a composition containing 4-AP and/or one or more derivatives of 4-AP is administered (e.g., orally administered) to a mammal (e.g., a human) identified as being in need of increased BMP-2 polypeptide expression, the mammal can have a disease, disorder, or condition characterized by a reduced level of one or more BMP-2 polypeptides. In some cases, a reduced level of BMP-2 polypeptide can refer to a level that is less than 10 μg/mL in a human. In some cases, a mammal having a disease, disorder, or condition characterized by a reduced level of one or more BMP-2 polypeptides can have bone loss (or can be identified as being likely to experience bone loss). Examples of diseases, disorders, and conditions that are characterized by a reduced level of one or more BMP-2 polypeptides and can be treated as described herein (e.g., by administering a composition containing 4-AP and/or one or more derivatives of 4-AP) include, without limitation, osteoporosis, bone cancers, bone fractures (e.g., insufficiency fractures), congenital bone diseases, degenerative bone diseases, dietary bone insufficiencies, and environmental exposure bone insufficiencies (e.g., bone insufficiencies caused by toxic exposure such as lead poisoning).

In some cases, methods described herein also can include identifying the mammal as having a disease, disorder, or condition characterized by a reduced level of one or more BMP-2 polypeptides. Examples of methods for identifying a mammal as having a disease, disorder, or condition characterized by a reduced level of one or more BMP-2 polypeptides include, without limitation, detecting a level of one or more BMP-2 polypeptides in a sample obtained from the mammal or identifying the mammal as having bone loss. Examples of methods for identifying a mammal as having bone loss include, without limitation, bone density tests and imaging techniques (e.g., X-rays, bone densitometry, micro-computed tomography (microCT), and ultrasound), and mechanical testing to determine the proportion of mineral in bones. Once identified as having a disease, disorder, or condition characterized by a reduced level of one or more BMP-2 polypeptides, the mammal can be administered or instructed to self-administer a composition described herein (e.g., a composition containing 4-AP and/or one or more derivatives of 4-AP).

In some cases, methods described herein also can include detecting a level of one or more BMP-2 polypeptides in a sample obtained from a mammal (e.g., a human). For example, a level of one or more BMP-2 polypeptides can be detected in a sample obtained from a mammal (e.g., a human) that has been administered a composition described herein (e.g., a composition containing 4-AP and/or one or more derivatives of 4-AP) before or after (or both before and after) that composition was administered to the mammal.

Any appropriate sample can be obtained from a mammal (e.g., a human) to detect a level of expression of one or more BMP-2 polypeptides. In some cases, a sample can be a biological sample. In some cases, a sample can contain one or more biological molecules (e.g., nucleic acids such as DNA and RNA, polypeptides, carbohydrates, lipids, hormones, and/or metabolites). In some cases, a sample can be a fluid sample. In some cases, a sample can be a tissue sample. Examples of samples that can be assessed as described herein include, without limitation, urine, whole blood, serum, plasma, bone tissue, bone marrow, lymph, saliva, and cerebrospinal fluid (CSF). A biological sample can be a fresh sample or a fixed sample (e.g., a formaldehyde-fixed sample or a formalin-fixed sample). In some cases, a biological sample can be a processed sample (e.g., to isolate or extract one or more biological molecules). In some cases, a sample can be from a mammal (e.g., a human) to be treated as described herein. In some cases, a sample can be from a mammal (e.g., a human) that is genetically related to a mammal to be treated as described herein.

Any appropriate method can be used to detect a level of expression of one or more BMP-2 polypeptides within a sample (e.g., a sample obtained from a mammal such as a human). In some cases, a level of expression of one or more BMP-2 polypeptides within a sample can be determined by detecting a level of one or more BMP-2 polypeptides in the sample. For example, immunoassays (e.g., immunohistochemistry (IHC) techniques, western blotting techniques, and enzyme-linked immunoassay (ELISA) assays) and mass spectrometry (MS) techniques (e.g., proteomics-based MS assays, targeted quantification-based MS assays, and mass spectrometry techniques including high performance liquid chromatography (HPLC-MS)) can be used to determine a level of one or more BMP-2 polypeptides in a sample. When an immunoassay is used to determine a level of one or more BMP-2 polypeptides in a sample, the immunoassay can use any appropriate antibody. Examples of antibodies that can be used in an immunoassay to determine a level of one or more BMP-2 polypeptides in a sample include, without limitation, human BMP-2 antibody MAB3551 (R&D systems) and EPR20807 (Abcam #ab214821). In some cases, a level of expression of one or more BMP-2 polypeptides within a sample can be determined by detecting a level of mRNA encoding a BMP-2 polypeptide in the sample. For example, polymerase chain reaction (PCR)-based techniques such as quantitative reverse transcription (RT)-PCR (qPCR) techniques, in situ hybridization techniques, northern blot assays, nuclease protection assays, and microarrays can be used to determine a level of mRNA encoding a BMP-2 polypeptide in a sample.

A sample can be obtained from a mammal (e.g., a human) at any time. In some cases, a sample can be obtained from a mammal before or after (or both before and after) the mammal has been administered a composition described herein (e.g., a composition containing 4-AP and/or one or more derivatives of 4-AP). For example, a sample can be obtained from mammal (e.g., a human) within 72 hours of the mammal having been administered a composition described herein (e.g., a composition containing 4-AP and/or one or more derivatives of 4-AP). For example, a sample can be obtained from mammal (e.g., a human) at least 0.5 hours after the mammal has been administered a composition described herein (e.g., a composition containing 4-AP and/or one or more derivatives of 4-AP).

In certain instances, a level of one or more BMP-2 polypeptides within a mammal (e.g., within a sample obtained from a mammal) can be detected at different time points over a course of a treatment with a composition described herein (e.g., a composition containing 4-AP and/or one or more derivatives of 4-AP). For example, two or more (e.g., two, three, four, five, six, or more) samples can be obtained from a mammal at different time point over the course of treatment.

A composition described herein (e.g., a composition containing 4-AP and/or one or more derivatives of 4-AP) can be administered (e.g., orally administered) to a mammal (e.g., a human) identified as being in need of increased BMP-2 polypeptide expression, to increase a level of one or more BMP-2 polypeptides in any type of bone. In some cases, the methods and materials provided herein can be used to increase a level of one or more BMP-2 polypeptides in cortical bone. In some cases, the methods and materials provided herein can be used to increase a level of one or more BMP-2 polypeptides in cancellous bone. Examples of types of bones that can be affected by a disease, disorder, or condition characterized by a reduced level of one or more BMP-2 polypeptides and can be treated as described herein include, without limitation, long bones, short bones, flat bones, irregular bones, sesamoid bones, and ossifications.

A composition described herein (e.g., a composition containing 4-AP and/or one or more derivatives of 4-AP) can be administered (e.g., orally administered) to a mammal (e.g., a human) identified as being in need of increased BMP-2 polypeptide expression, to increase a level of one or more BMP-2 polypeptides in any bone within a mammal (e.g., a bone in any location within a mammal). In some cases, the methods and materials provided herein can be used to increase a level of one or more BMP-2 polypeptides in the spine of a mammal. In some cases, the methods and materials provided herein can be used to increase a level of one or more BMP-2 polypeptides in an arm of a mammal. In some cases, the methods and materials provided herein can be used to increase a level of one or more BMP-2 polypeptides in a leg of a mammal.

A composition described herein (e.g., a composition containing 4-AP and/or one or more derivatives of 4-AP) can include 4-AP and/or any appropriate derivative(s) of 4-AP. Examples of derivatives of 4-AP that can be included in a composition described herein include, without limitation, 3,4-diaminopyridine, 3-hydroxy-4-aminopyridine, N-(4-pyridyl)-t-butyl carbamate, N-(4-pyridyl)ethyl carbamate, N-(4-pyridyl) methyl carbamate, and N-(4-pyridyl) isopropyl carbamate. In some cases, 4-AP and/or one or more derivatives of 4-AP can have a structure according to Formula I:

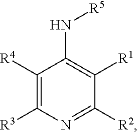

where R¹, R², R³, R⁴, and R⁵ are each independently selected from hydrogen (H), halogen (e.g., fluorine (F), chlorine (Cl), bromine (Br) or iodine (I)), amine, hydroxyl (e.g., —OH), alkoxy (e.g., —OAk), carboxyl (e.g., —CO₂H), alkoxycarbonyl (e.g., —C(O)—OAk), or alkyl (e.g., C1-6 alkyl or Ak). For example, R¹, R², R³, R⁴, and R⁵ can all be hydrogen. Examples of amine include —NR^{N1}R^{N2}, in which each of R^{N1} and R^{N2} is, independently, H or optionally substituted alkyl, or R^{N1} and R^{N2}, taken together with the nitrogen atom to which each are attached, form a heterocyclyl group. Examples of alkyl or Ak include a branched or unbranched saturated hydrocarbon group of 1 to 24 carbon atoms (C1-24), such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C3-24 cycloalkyl) or acyclic. The alkyl group can be branched or unbranched. The alkyl group can also be substituted or unsubstituted. Substitutions for alkyl can include a halogen (e.g., any herein), hydroxyl, alkoxy, carboxyl, or amine.

In some cases, 4-AP or a derivative thereof can be a potassium channel blocker. In some cases, 4-AP or a derivative thereof can be a calcium channel agonist. In some cases, 4-AP or a derivative thereof can be electrically active. In some cases, 4-AP or a derivative thereof can be in the form of a free base. In some cases, 4-AP or a derivative thereof can be in the form of a salt (e.g., pharmaceutically acceptable salt). When 4-AP or a derivative thereof is in the form of a salt, the salt can include any appropriate acid (e.g., an organic acid or an inorganic acid). Examples of acids that can be used to form a salt with 4-AP or a derivative thereof include, without limitation, hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, and mandelic acid.

In some cases, 4-AP and/or one or more derivatives of 4-AP can be as described elsewhere (see, e.g., U.S. Patent Application Publication No. 2018/0271847, U.S. Pat. No. 9,993,429, and International Patent Application Publication WO 2021/150773).

A composition described herein (e.g., a composition containing 4-AP and/or one or more derivatives of 4-AP) can include any appropriate amount of 4-AP and/or one or more derivatives of 4-AP. In some cases, a composition described herein can include from about 0.1 μM to about 150 μM (e.g., from about 0.1 μM to about 125 μM, from about 0.1 μM to about 100 μM, from about 0.1 μM to about 75 μM, from about 0.1 μM to about 50 μM, from about 0.1 μM to about 30 μM, from about 0.1 μM to about 20 μM, from about 0.1 μM to about 10 μM, from about 0.1 μM to about 5 μM, from about 0.1 μM to about 1 μM, from about 1 μM to about 150 μM, from about 5 μM to about 150 μM, from about 10 μM to about 150 μM, from about 20 μM to about 150 μM, from about 30 μM to about 150 μM, from about 50 μM to about 150 μM, from about 75 μM to about 150 μM, from about 100 μM to about 150 μM, from about 125 μM to about 150 μM, from about 1 μM to about 125 μM, from about 5 μM to about 100 μM, from about 10 μM to about 75 μM, from about 25 μM to about 50 μM, from about 5 μM to about 25 M, from about 25 μM to about 50 μM, from about 50 μM to about 75 μM, from about 75 μM to about 100 μM, or from about 100 μM to about 125 μM) of 4-AP and/or one or more derivatives of 4-AP. In some cases, a composition described herein can include from about 0.01% to about 99% (e.g., from about 0.01% to about 90%, from about 0.01% to about 80%, from about 0.01% to about 70%, from about 0.01% to about 60%, from about 0.01% to about 50%, from about 0.01% to about 40%, from about 0.01% to about 30%, from about 0.01% to about 20%, from about 0.01% to about 10%, from about 0.01% to about 5%, from about 0.01% to about 1%, from about 1% to about 99%, from about 5% to about 99%, from about 10% to about 99%, from about 20% to about 99%, from about 30% to about 99%, from about 40% to about 99%, from about 50% to about 99%, from about 60% to about 99%, from about 70% to about 99%, from about 80% to about 99%, from about 90% to about 99%, from about 10% to about 90%, from about 20% to about 80%, from about 30% to about 70%, from about 40% to about 60%, from about 10% to about 30%, from about 30% to about 50%, from about 50% to about 70%, or from about 70% to about 90%) of 4-AP and/or one or more derivatives of 4-AP.

In some cases, a composition described herein (e.g., a composition containing 4-AP and/or one or more derivatives of 4-AP) can include one or more pharmaceutically acceptable carriers (additives), excipients, and/or diluents. Examples of pharmaceutically acceptable carriers, excipients, and diluents that can be used in a composition described herein include, without limitation, saline (e.g., phosphate-buffered saline (PBS)), sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., poly(lactic-co-glycolic acid), polyethylene glycol, polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition described herein (e.g., a composition containing 4-AP and/or one or more derivatives of 4-AP) can be administered to a mammal in need thereof (e.g., a mammal having bone loss) locally or systemically. In some cases, a compositions described herein can be administered locally. For example, a composition described herein can be administered locally by injection directly into, around, and/or near an area of bone loss on a mammal (e.g., a human). In some cases, a composition described herein can be administered systemically. For example, a composition described herein can be designed for oral or parenteral (including intraperitoneal, subcutaneous, intramuscular, intravenous, and intradermal) administration to a mammal having bone loss. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition described herein can be formulated for parenteral administration (e.g., intraperitoneal injection or intravenous injection).

Any appropriate amount can be administered to a mammal to treat the mammal as described herein. For example, an effective amount of 4-AP and/or one or more derivatives of 4-AP that can be administered to a mammal (e.g., a human) can be any amount that increases a level of one or more BMP-2 polypeptides (e.g., can treat a disease, disorder, or condition characterized by a reduced level of one or more BMP-2 polypeptides such as bone loss) in a mammal without producing significant toxicity to the mammal. In some cases, an effective amount of 4-AP and/or one or more derivatives of 4-AP can be from about 0.05 milligrams per kilogram body weight (mg/kg) to about 1 mg/kg (e.g., from about 0.05 mg/kg to about 0.9 mg/kg, from about 0.05 mg/kg to about 0.8 mg/kg, from about 0.05 mg/kg to about 0.7 mg/kg, from about 0.05 mg/kg to about 0.6 mg/kg, from about 0.05 mg/kg to about 0.5 mg/kg, from about 0.05 mg/kg to about 0.4 mg/kg, from about 0.05 mg/kg to about 0.3 mg/kg, from about 0.05 mg/kg to about 0.2 mg/kg, from about 0.05 mg/kg to about 0.1 mg/kg, from about 0.1 mg/kg to about 1 mg/kg, from about 0.2 mg/kg to about 1 mg/kg, from about 0.3 mg/kg to about 1 mg/kg, from about 0.4 mg/kg to about 1 mg/kg, from about 0.5 mg/kg to about 1 mg/kg, from about 0.6 mg/kg to about 1 mg/kg, from about 0.7 mg/kg to about 1 mg/kg, from about 0.8 mg/kg to about 1 mg/kg, from about 0.9 mg/kg to about 1 mg/kg, from about 0.1 mg/kg to about 0.9 mg/kg, from about 0.2 mg/kg to about 0.8 mg/kg, from about 0.3 mg/kg to about 0.7 mg/kg, from about 0.4 mg/kg to about 0.6 mg/kg, from about 0.1 mg/kg to about 0.3 mg/kg, from about 0.2 mg/kg to about 0.4 mg/kg, from about 0.3 mg/kg to about 0.5 mg/kg, from about 0.4 mg/kg to about 0.6 mg/kg, from about 0.5 mg/kg to about 0.7 mg/kg, from about 0.6 mg/kg to about 0.8 mg/kg, or from about 0.7 mg/kg to about 0.9 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the disease, disorder, or condition characterized by a reduced level of one or more BMP-2 polypeptides (e.g., bone loss) may require an increase or decrease in the actual effective amount administered.

The frequency of administration of a composition described herein (e.g., a composition containing 4-AP and/or one or more derivatives of 4-AP) can be any frequency that increases a level of one or more BMP-2 polypeptides (e.g., can treat a disease, disorder, or condition characterized by a reduced level of one or more BMP-2 polypeptides such as bone loss) in a mammal without producing significant toxicity to the mammal. For example, the frequency of administration can be from about once a day to about once a week, from about once a week to about once every two months, from about once every two weeks to about once every six weeks, or from about once every three weeks to about once a month (e.g., once every four weeks). The frequency of administration can remain constant or can be variable during the duration of treatment. A course of treatment with a composition described herein can include rest periods. For example, a composition described herein can be administered once a month over a six-month period followed by a rest period (e.g., a one or two month rest period), and such a regimen can be repeated multiple times. As with the effective amount, various factors can influence the actual frequency of administration used for a particular application. For example, the effective amount, duration of treatment, use of multiple treatment agents, route of administration, and severity of the bone loss may require an increase or decrease in administration frequency.

An effective duration for administering a composition described herein (e.g., a composition containing 4-AP and/or one or more derivatives of 4-AP) can be any duration that increases a level of one or more BMP-2 polypeptides (e.g., can treat a disease, disorder, or condition characterized by a reduced level of one or more BMP-2 polypeptides such as bone loss) in a mammal without producing significant toxicity to the mammal. For example, the effective duration can vary from several days to several weeks, months, or years. In some cases, the effective duration for the treatment of bone loss can range in duration from about one month to about 6 months. Multiple factors can influence the actual effective duration used for a particular treatment. For example, an effective duration can vary with the frequency of administration, effective amount, use of multiple treatment agents, route of administration, and severity of the bone loss being treated.

In some cases, the methods and materials described herein can be used as the sole active agent used to increase a level of one or more BMP-2 polypeptides in a mammal (e.g., to treat a mammal such as a human having a disease, disorder, or condition characterized by a reduced level of one or more BMP-2 polypeptides such as bone loss). For example, a composition containing 4-AP and/or one or more derivatives of 4-AP can be used as the sole active agent(s) used to increase a level of one or more BMP-2 polypeptides in a mammal (e.g., a human).

In some cases, methods described herein also can include administering to a mammal (e.g., a human) identified as having a disease, disorder, or condition characterized by a reduced level of one or more BMP-2 polypeptides (e.g., identified as having bone loss or as being likely to experience bone loss) one or more (e.g., one, two, three, four, five or more) additional agents used to increase a level of one or more BMP-2 polypeptides and/or to treat bone loss in addition to a composition described herein (e.g., a composition containing 4-AP and/or one or more derivatives of 4-AP). Examples of agents that can be used to increase a level of one or more BMP-2 polypeptides include, without limitation, recombinant BMP-2 polypeptides such as recombinant human protein (rhBMP-2). Examples of agents that can be used to treat bone loss include, without limitation, alendronate (e.g., BINOSTO® and FOSAMAX®), risedronate (e.g., ACTONEL® and ATELVIA®), ibandronate (e.g., BONIVA®), zoledronic acid (e.g., RECLAST® and ZOMETA®), denosumab (e.g., PROLIA® and XGEVA®), estrogen, raloxifene (e.g., EVISTA®), teriparatide (e.g., FORTEO®), abaloparatide (e.g., TYMLOS®), and romosozumab (EVENITY®). In cases where a mammal having a disease, disorder, or condition characterized by a reduced level of one or more BMP-2 polypeptides (e.g., bone loss) is treated with a composition described herein and is treated with one or more additional agents used to increase a level of one or more BMP-2 polypeptides and/or to treat bone loss, the additional agent(s) can be administered at the same time or independently. For example, the additional agent(s) can be formulated into a composition containing 4-AP and/or one or more derivatives of 4-AP to form a single composition. In some cases, a composition described herein can be administered first, and the one or more additional agents administered second, or vice versa.

The invention will be further described in the following examples, which do not limit the scope of the invention described in the claims.

### EXAMPLES

### Example 1: 4-AP and BMP-2 Expression in Human Bone Marrow Mesenchymal Stem Cells

Human bone marrow mesenchymal stem cells (hBMSC) were cultured in growth medium supplemented with 4-AP for 3, 7, and 14 days. Total RNA was isolated from the cells at the end of treatment, converted to cDNA, and the gene expression of bone morphogenetic protein-2 (BMP-2) was analyzed by quantitative real-time PCR.

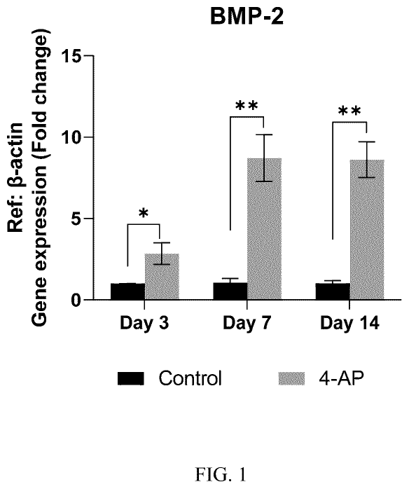

Gene expression by quantitative real-time PCR: Quantitative real-time PCR was carried out to determine gene expression of BMP-2 by hBMSCs incubated in Dulbecco's modified Eagle's medium with 10% fetal bovine serum (FBS) and penicillin/streptomycin (100 U/mL penicillin and 100 mg/mL streptomycin) at a humidified atmosphere of 5% CO₂ under 37° C. Cell cultures were supplemented with 1000 μM 4-AP for 3, 7, and 14 days. Total RNA was isolated from the cells and converted to cDNA using verso cDNA synthesis kit (Thermo Fischer Scientific). The cDNA obtained was analyzed in a quantitative real-time PCR (FIG. 1).

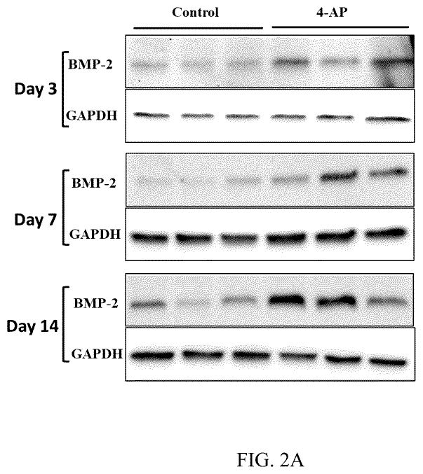

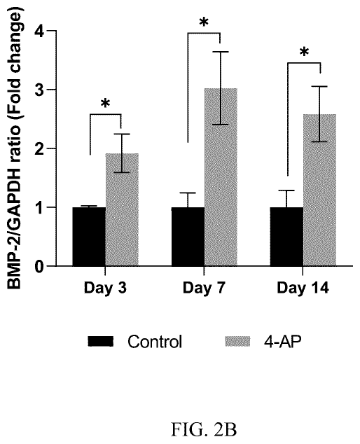

Protein level by western blot: Total protein was extracted from hBMSCs with RIPA lysis buffer at the end of 3, 7, and 14 days of 4-AP treatment. The protein concentrations of cell lysates were determined using Pierce BCA Protein Assay kit (Thermo Fischer Scientific). Total cell lysates containing equal amounts of protein were denatured with SDS reducing buffer. The denatured proteins were separated on 10% polyacrylamide gel and transferred to a PVDF membrane. The blots were blocked and probed with primary and secondary antibodies, followed by visualization of bands by chemiluminescence peroxidase substrate. The BMP-2 band intensities were analyzed by ImageJ software (FIGS. 2A and 2B).

These results demonstrate that a composition containing 4-AP and/or one or more derivatives of 4-AP can be administered to a mammal (e.g., a human) to increase a level of BMP-2 polypeptides within the mammal, thereby having the ability to treat bone loss, strengthen bone, and promote bone healing within a mammal.

### Example 2: 4-AP and BMP-2 Expression in Human Fetal Osteoblasts

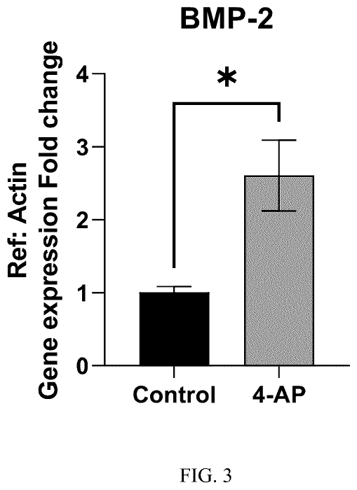

Human fetal osteoblasts (hFOB) were cultured in growth medium supplemented with 4-AP for 3 days. Total RNA was isolated from the cells at the end of treatment, converted to cDNA, and the gene expression of bone morphogenetic protein-2 (BMP-2) was analyzed by quantitative real-time PCR (FIG. 3).

These results demonstrate that a composition containing 4-AP and/or one or more derivatives of 4-AP can be administered to a mammal (e.g., a human) to increase a level of BMP-2 polypeptides within the mammal, thereby having the ability to treat bone loss, strengthen bone, and promote bone healing within a mammal.

### Example 3: 4-AP and Bone Mineral Density in Animal Models

Ten-week-old male C57/BL6J mice were purchased from the Jackson laboratory (Bar Harbor, ME, USA) and were maintained in a 12-h light, 12-h dark cycle at 21.1° C. to 22.8° C. After acclimatization, all mice were randomly divided in to two groups: 1) control and 2) 4-AP.

A mid diaphysis tibial fracture was performed in ten-week-old male mice. After the mice were anesthetized with ketamine/xylazine, a 5 mm longitudinal incision will be made at the front of the proximal tibia. The mid diaphysis of the tibia will be identified, and an osteotomy will be created with a No. 11 scalpel blade preventing any comminution. A 27G hypodermic needle will be introduced into the tibial canal for intramedullary fixation. Buprenorphine was given as post-operative analgesia. X-ray images were acquired post-operatively and mice received saline or 4-AP on the same day. Control group received 100 μl of saline, and a 4-AP group received 40 μg/mouse/daily 4-AP (1.6 mg/kg) in saline intraperitoneally until day 21 post-surgery.

For micro-CT analysis, tibia of control and 4-AP mice were dissected of soft tissue and fixed in neutral buffered formalin. μCT scanning was conducted using a Scanco vivaCT 40. For callus analysis, tibia was scanned for a region spanning from femoral condyle growth plate to 3 mm distal from the callus center. Callus area from control and 4-AP mice tibia were assessed for bone mineral density (BMD) and percent bone volume per total volume (% BV/TV).

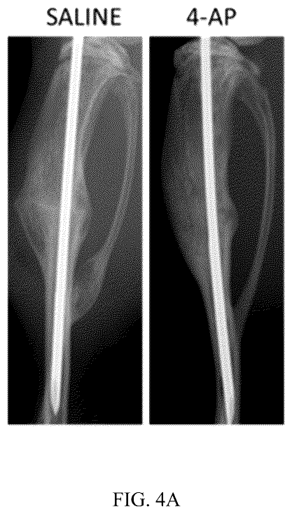

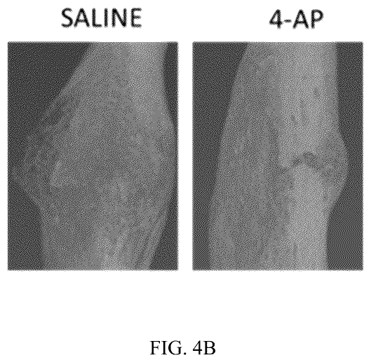

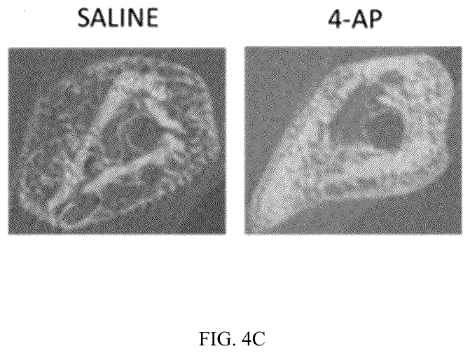

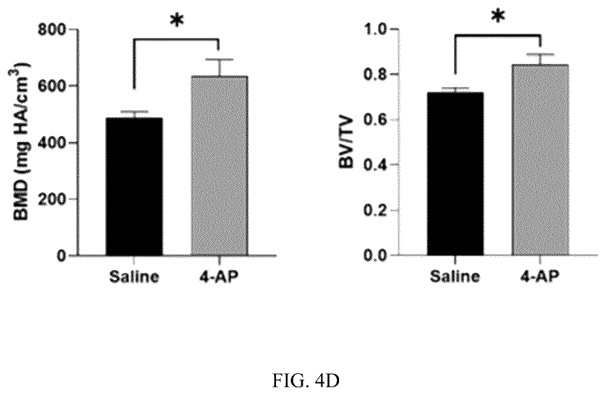

Tibial fracture was performed in C57BL/6J mice, which were then treated with 4-AP for 21 days for the evaluation of fracture healing parameters. The bone mineral density and bone volume density were determined by micro-CT analysis at the end of 21 days 4-AP treatment. The results showed a higher bone mineral density and bone volume density within the callus area of 4-AP treated mice compared to saline treated controls (FIGS. 4A to 4D).

These results demonstrate that a composition containing 4-AP and/or one or more derivatives of 4-AP can be administered to a mammal (e.g., a human) to increase a level of BMP-2 polypeptides within the mammal, thereby having the ability to treat bone loss, strengthen bone, and promote bone healing within a mammal.

### Example 4: Increasing BMP-2 Polypeptide Expression

A human in need of treatment of a disease, disorder, or condition associated with reduced BMP-2 polypeptides is administered or self-administers a composition containing 4-AP and/or one or more derivatives of 4-AP. The administered composition containing 4-AP and/or one or more derivatives of 4-AP can increase a level of one or more BMP-2 polypeptides in the human (e.g., can increase BMP-2 polypeptide expression by cells within the human).

### Example 5: Treating Bone Loss

A human identified as having bone loss (e.g., bone loss associated with a reduced BMP-2 polypeptides) is administered or self-administers a composition containing 4-AP and/or one or more derivatives of 4-AP. The administered composition containing 4-AP and/or one or more derivatives of 4-AP can treat the bone loss. In some cases, the administered composition containing 4-AP and/or one or more derivatives of 4-AP can increase the bone mineral density (BMD) of one or more bones within the mammal. In some cases, the administered composition containing 4-AP and/or one or more derivatives of 4-AP can increase the bone mineral content (BMC) of one or more bones within the mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for increasing a level of a bone morphogenetic protein 2 (BMP-2) polypeptide in a mammal, wherein said method comprises administering a composition comprising 4-AP or one or more derivatives of 4-AP to said mammal.

2. The method of claim 1, wherein said mammal is a human.

3. The method of claim 2, wherein said human is identified as being in need of increased BMP-2 polypeptide expression.

4. The method of claim 1, wherein said administering comprises a systemic administration.

5. The method of claim 4, wherein said systemic administration is an oral administration.

6. The method of claim 1, wherein said mammal has a disease, disorder, or condition associated with reduced BMP-2 polypeptide expression.

7. The method of claim 6, wherein said disease, disorder, or condition associated with reduced BMP-2 polypeptide expression comprises bone loss.

8. The method of claim 6, wherein said disease, disorder, or condition is selected from the group consisting of osteoporosis, a bone cancer, a bone fracture, a congenital bone disease, a degenerative bone disease, a dietary bone insufficiency, and an environmental exposure bone insufficiency.

9. A method for treating a mammal having a disease, disorder, or condition associated with reduced BMP-2 polypeptide expression, wherein said method comprises:
administering a composition comprising 4-AP or one or more derivatives of 4-AP to said mammal; and
detecting an increased level of a BMP-2 polypeptide in a sample obtained from said mammal.

10. The method of claim 9, wherein said increased level of said BMP-2 polypeptide comprises a serum level that is greater than 10 μg/mL.

11. The method of claim 9, wherein said sample is selected from the group consisting of urine, whole blood, serum, plasma, bone tissue, bone marrow, lymph, saliva, and cerebrospinal fluid (CSF) samples.

12. The method of claim 9, wherein said sample is obtained at least 0.5 hours after said administering of said composition comprising 4-AP or one or more derivatives of 4-AP to said mammal.

13. The method of claim 9, wherein said mammal is a human.

14. The method of claim 9, wherein said administering comprises a systemic administration.

15. The method of claim 14, wherein said systemic administration is oral administration.

16. The method of claim 13, wherein said human is identified as having said disease, disorder, or condition associated with reduced BMP-2 polypeptide expression.

17. The method of claim 16, wherein said disease, disorder, or condition associated with reduced BMP-2 polypeptide expression comprises bone loss.

18. The method of claim 17, wherein said disease, disorder, or condition is selected from the group consisting of osteoporosis, a bone cancer, a bone fracture, a congenital bone disease, a degenerative bone disease, a dietary bone insufficiency, and an environmental exposure bone insufficiency.

19. A composition comprising 4-AP or one or more derivatives of 4-AP for use in the preparation of a medicament to increase BMP-2 polypeptide expression in a mammal or for use in increasing BMP-2 polypeptide expression in a mammal.

20. The composition of claim 19, wherein said mammal is a human and optionally wherein said human is identified as being in need of increased BMP-2 polypeptide expression.
